(19) United States
(12) Patent Application Publication
(10) Pub. No.: US 2016/0143619 A1
(43) Pub. Date: May 26, 2016

(54) ULTRASONIC PROBE HAVING A PLURALITY OF ARRAYS CONNECTED IN PARALLEL STRUCTURE AND ULTRASONIC IMAGE DIAGNOSING APPARATUS INCLUDING SAME

(71) Applicant: ALPINION MEDICAL SYSTEMS CO., LTD., Seoul (KR)

(72) Inventors: Byung-Kuk BAE, Daegu (KR); Su-Sung LEE, Yongin-si (KR)

(73) Assignee: ALPINION MEDICAL SYSTEMS CO., LTD., Seoul (KR)

(21) Appl. No.: 14/901,602

(22) PCT Filed: Jun. 28, 2013
(86) PCT No.: PCT/KR2013/005781

§ 371 Date: Dec. 28, 2015

(51) Int. Cl. A61B 8/00; A61B 8/08

(52) U.S. Cl. CPC A61B 8/4494; A61B 8/4444; A61B 8/54; A61B 8/5207; A61B 8/461

(57) ABSTRACT

An ultrasonic probe having a plurality of arrays connected in a parallel structure and an ultrasonic image diagnosing apparatus including the same are disclosed. The ultrasonic probe according to one embodiment of the present invention comprises: a first array; a second array connected in parallel to the first array in the elevation direction of the first array and having a focal distance different from that of the first array by having width different from that of the first array in the elevation direction; and a switch or a multiplexer for selecting an array to be activated.

### TECHNICAL FIELD

The present invention relates to an imaging diagnostic technology, and more specifically, to an ultrasonic imaging diagnostic technology.

### BACKGROUND ART

Electronic engineering and signal processing recently develops rapidly; and particularly, the digital signal processing technology greatly affects a field of an imaging diagnostic apparatus. The imaging apparatus is like a flower of a medical diagnostic apparatus in that an internal body can be seen without cutting the internal body. For such an imaging diagnostic apparatus, an X-ray diagnostic apparatus, a magnetic resonance imaging (MRI) diagnostic apparatus, etc., are used, which each have their own advantages and disadvantages. Among them, an ultrasonic imaging diagnostic apparatus can perform a real-time diagnosis and have an advantage of a low price. The ultrasonic imaging diagnostic apparatus has become essential in all medical fields, e.g., internal medicine, obstetrics & gynecology, pediatrics, urology, ophthalmology, radiology, so that its demand increases.

The ultrasonic imaging diagnostic apparatus includes an ultrasonic probe that transmits an ultrasonic signal to an object and receives an ultrasonic echo signal reflected from the object. The ultrasonic probe may acquire images, of which resolution may be different from each other according to properties of an operation frequency.

For example, if a transmission ultrasonic beam has high-frequency properties, an ultrasonic beam's focusing properties are good at a region that is close to the probe, i.e., a shallow region of the object, so that images with high resolution may be acquired. On the other hand, it is relatively difficult for the ultrasonic beam to penetrate a region that is far away from the probe, i.e., a deep region of the object, thereby deteriorating transmission focusing properties, so that the resolution is lowered.

In contrast, if a transmission ultrasonic beam has low-frequency properties, a region close to the probe, i.e., a shallow region of the object, has lower resolution, compared to the transmission ultrasonic beam having high-frequency properties, whereas it is relatively easy for the ultrasonic beam to penetrate a region far away from the probe, i.e., a deep region of the object, thereby acquiring images with improved resolution. Thus, an ultrasonic probe is required, which can acquire images with the highest qualities appropriate for various properties of an object.

### TECHNICAL PROBLEM

According to an exemplary embodiment, proposed are an ultrasonic probe, which includes a plurality of arrays connected to each other in parallel, and an ultrasonic imaging diagnostic apparatus, which includes the same, so as to acquire optimum images regardless of properties of an object.

### TECHNICAL SOLUTION

In an general aspect, an ultrasonic probe includes: a first array; a second array to be connected to the first array in parallel in an elevation direction, and have a focal length that is different from a focal length of the first array due to a width that is different from a width of the first array in an elevation direction; and a switch to select one of the first and second arrays and activate the selected array.

The first array may be a near-field array with a width h1 and a focal length L1, and the second array may be a far-field array with a width h2 and a focal length L2, where h1 is smaller than h2 and L1 is smaller than L2.

The switch may operate the first array at a high frequency, and the second array at a low frequency. The switch may in response to an array drive signal being applied to a first access point, activate the first array, and in response to the array drive signal being applied to a second access point, activate the second array.

Each array may have a concave surface that is positioned in an axial direction in which beams proceed. The ultrasonic probe may further include an acoustic lens to focus, on an inside of an object, an ultrasonic signal generated from the array activated by the switch, wherein the acoustic lens may a plane.

In another general aspect, an ultrasonic probe includes: a first array; a second array to be connected to the first array in parallel in an elevation direction, and have a focal length that is different from a focal length of the first array due to a width that is different from a width of the first array in an elevation direction; and a multiplexer to simultaneously activate the first and second arrays and control the activated arrays to focus ultrasonic signals on each array.

The first array may be a near-field array with a width h1 and a focal length L1, and the second array may be a far-field array with a width h2 and a focal length L2, where h1 is smaller than h2 and L1 is smaller than L2. The multiplexer may operate the first array at a high frequency, and the second array at a low frequency.

Each array may have a concave surface that is positioned in an axial direction in which beams proceed. The ultrasonic probe may further include an acoustic lens to focus, on an inside of an object, the ultrasonic signal generated from the array activated by the multiplexer, wherein the acoustic lens may be a plane.

The ultrasonic probe may further include an acoustic lens to focus, on an inside of an object, the ultrasonic signal generated from the array activated by the multiplexer, wherein the acoustic lens may be a plane.

In another general aspect, an ultrasonic imaging diagnostic apparatus includes: an ultrasonic probe, which includes a plurality of arrays each having different focal lengths due to different widths in an elevation direction, and which includes s a switch to select one of the plurality of arrays and activate the selected array; a transceiver to transceive an ultrasonic signal to or from an object through the array selected and activated by the ultrasonic probe; an image processor to in response to a reflected ultrasonic signal being received from the object by the transceiver, generate a displayable image by using the received reflected ultrasonic signal; and a display to display the image generated by the image processor.

The ultrasonic probe may include a near-field array with a width h1 and a focal length L1, and a far-field array with a width h2 and a focal length L2, where h1 is smaller than h2, and L1 is smaller than L2.

In another general aspect, an ultrasonic imaging diagnostic apparatus includes: an ultrasonic probe, which includes a plurality of arrays each having different focal lengths due to different widths in an elevation direction, and which includes a multiplexer to simultaneously activate the plurality of arrays and control the activated arrays to focus ultrasonic signals on each array; a transceiver to transceive an ultrasonic signal to or from an object through the array activated by the ultrasonic probe; an image processor to in response to a reflected ultrasonic signal being received from the object by the transceiver, generate a displayable image by using the received reflected ultrasonic signal; and a display to display the image generated by the image processor.

The ultrasonic probe may include a near-field array with a width h1 and a focal length L1, and a far-field array with a width h2 and a focal length L2, where h1 is smaller than h2, and L1 is smaller than L2.

### ADVANTAGEOUS EFFECTS

According to an exemplary embodiment, using a single probe, an optimum array is selected adaptively to an environment where the depth of a patient's target tissue changes, so that images with optimum qualities may be acquired. For example, regardless of a patient's circumstances in which the depth of the patient's target tissue changes, e.g., cases where the patient is fat, so that the ultrasonic waves need to penetrate deeply, and where the patient is slim, so that the ultrasonic waves need to penetrate shallowly, the optimum array is selected appropriately for each circumstance, thereby acquiring images with optimum qualities.

### DESCRIPTION OF DRAWINGS

FIG. 1 is a diagram in which a space axis of an ultrasonic probe according to an exemplary embodiment is defined.

FIGS. 2 and 3 are diagrams illustrating structures of an ultrasonic probe according to an exemplary embodiment.

FIG. 4 is a diagram illustrating focal lengths of arrays having different widths from each other.

FIG. 5 is a diagram illustrating a constitution of an ultrasonic probe having a switch according to an exemplary embodiment.

FIG. 6 is a diagram illustrating an operation principle of a switch in FIG. 5 according to an exemplary embodiment.

FIG. 7 is a diagram illustrating an ultrasonic probe having a multiplexer according to an exemplary embodiment.

FIG. 8 is a diagram illustrating images acquired in a case where a multiplexer in FIG. 7 according to an exemplary embodiment is used.

FIG. 9 is a diagram illustrating a constitution of an ultrasonic imaging diagnostic apparatus according to an exemplary embodiment.

### MODE FOR INVENTION

The following detailed description is provided to assist the reader in gaining a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness. In the following description, a detailed description of known functions and configurations incorporated herein will be omitted when it may obscure the subject matter with unnecessary detail. Before describing the exemplary embodiments, terms used throughout this specification are defined. These terms are defined in consideration of functions according to exemplary embodiments, and can be varied according to a purpose of a user or manager, or precedent and so on. Therefore, definitions of the terms should be made on the basis of the overall context.

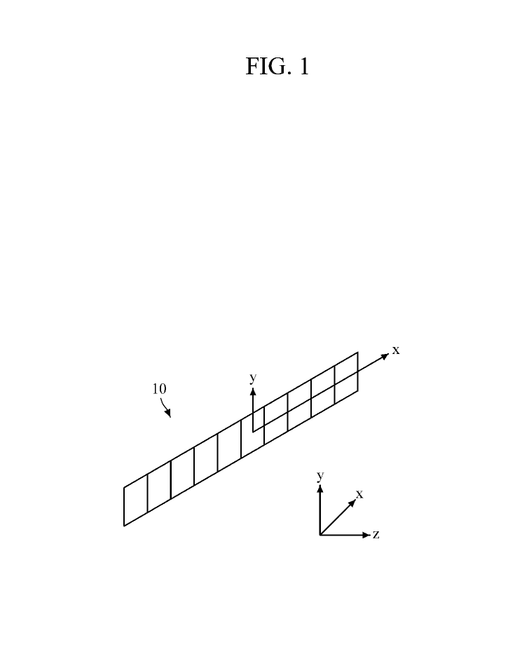

FIG. 1 is a diagram in which a space axis of an ultrasonic probe according to an exemplary embodiment is defined.

Referring to FIG. 1, a direction in which arrays, e.g., linear arrays, line up is defined as an azimuthal direction; a direction in which beams proceed, as an axial direction; and a direction that is orthogonal to these two directions, as an elevation direction. In a case of an ultrasonic probe composed of arrays, an azimuthal direction may be defined as an x axis; an axial direction, as a z axis; and an elevation direction, as a y axis. The following description is described based on a linear array of arrays, to which the form of the array is not limited.

In general, an array probe has arrays that are arranged in an azimuthal direction, in which focusing, adjustment, or a scan line movement, etc., through grouping the arrays may be electronically executed. However, the electronic focusing, the scan line movement, etc., are not possible in an elevation direction. Thus, the array probe includes an acoustic lens attached to the front end of the probe in an elevation direction, so that the array probe has a fixed focus.

A beam field is set by a fixed focus of an array probe, and by changing an operation frequency according to a bandwidth of the probe, its penetration and resolution may be changed, which may be then used. Nevertheless, since a circumstance of a patient, who is an object, may make big differences by the property of having a fixed focus, sometimes two or more probes are required. In such a case, if a low frequency is selected to increase the penetration, thereby reducing the resolution; and if a high frequency is selected to increase the resolution, thereby reducing the penetration.

Accordingly, the present invention provides a probe structure for acquiring an optimum image according to each circumstance of a patient not by using several probes but only using one probe. For example, an optimum array is selected adaptively to a patient's circumstances in which the patient's depth changes, such as cases where a patient is fat, so that ultrasonic waves need to penetrate deeply, and where a patient is slim, so that the ultrasonic waves need to penetrate shallowly, thereby acquiring images with the optimum qualities. In another example, the optimum array is selected adaptively to the circumstances where the patient's depth changes according to a direction in which an ultrasonic wave is transmitted toward a patient, such as cases of transmitting the ultrasonic wave toward the patient in an azimuthal direction and in front and rear directions, so that images with the optimum qualities may be acquired.

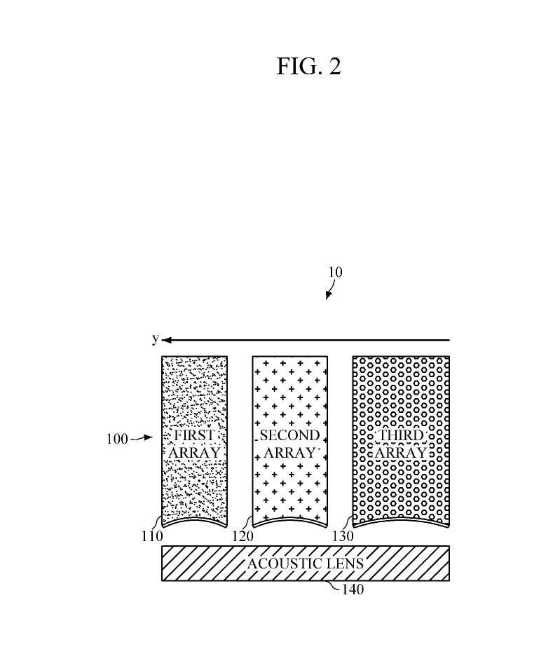

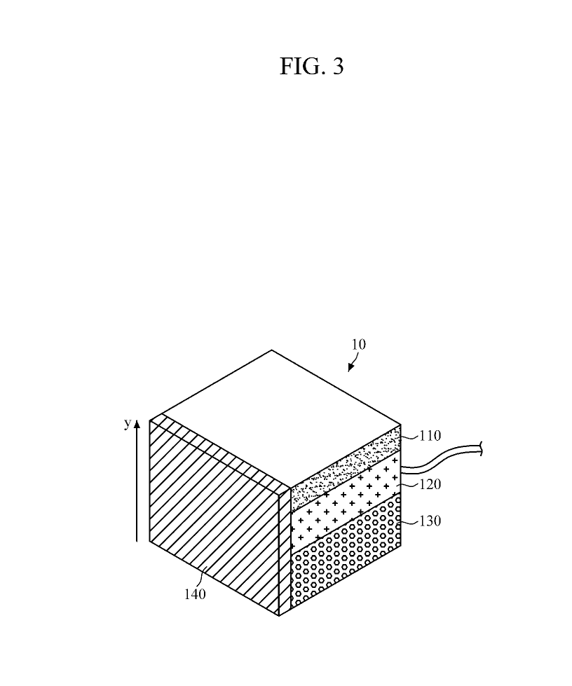

FIGS. 2 and 3 are diagrams illustrating structures of an ultrasonic probe according to an exemplary embodiment.

Referring to FIGS. 2 and 3, an ultrasonic probe includes a plurality of arrays 100 that is connected to each other in parallel in an elevation direction. Here, the plurality of arrays 100 has each different array width in an elevation direction. For example, as illustrated in FIG. 2, the plurality of arrays 100 are connected to each other in parallel in order of the width from thickest to thinner, i.e., in order of a third array 130, a second array 120, and a first array 110. However, the above-mentioned examples are only for the comprehension of the present invention, and if the ultrasonic probe only includes the arrays that have each different width in an elevation direction, the examples of its arrangement order or the total number of the arrays are not limited thereto and may change in various forms.

The first array 110, the second array 120, and the third array 130 includes elements, each of which includes a piezoelectric element, a backing layer, and a matching layer; and according to the examples of the present invention, a part in which the thickness of the array width is different from each other in an elevation direction is the piezoelectric element. The piezoelectric element performs a function of an interconversion between an electrical signal and an ultrasonic signal. The backing layer performs functions of absorbing an ultrasonic signal that, when energy of the piezoelectric element is excited in response to an electrical transmission signal, is immediately output from the piezoelectric element by the vibration of the piezoelectric element and then propagated in an opposite direction to the direction in which the ultrasonic signal has been transmitted. The matching layer performs a function of covering the piezoelectric element so as to reduce the acoustic impedance difference between the piezoelectric element and the object.

The first array 110, the second array 120, and the third array 130 may have concave surfaces that are positioned in an axial direction in which the beams proceed. In such a case, an acoustic lens 140 focuses, on the inside of each object, the ultrasonic signals, which have been generated, respectively, from the first array 110, the second array 120, and the third array 130. The acoustic lens 140 according to an exemplary embodiment may be a plane.

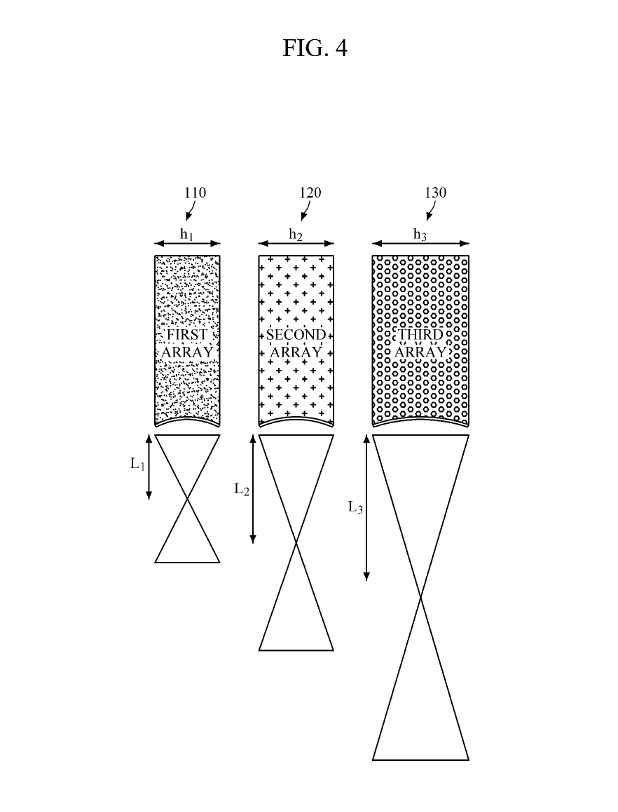

FIG. 4 is a diagram illustrating focal lengths of arrays having different widths from each other.

Referring to FIG. 4, since arrays each have widths that are different from each other in an elevation direction, their focal lengths are different from each other. For example, as illustrated in FIG. 4, the first array 110 is a near-field array, in which a width h1 is thin, so that a focal length L1 is short. On the other hand, the third array 130 is a far-field array, in which a width h3 is thick, so that a focal length L3 is long.

In one exemplary embodiment, the arrays each have different frequencies from each other as having widths that are different from each other in an elevation direction. If a piezoelectric element is thick due to its properties, properties of the low frequency are more shown, rather than the properties of the high frequency. For example, since the near-field array, i.e., the first array 110, has a thin width h1, an operation frequency f1 is high. On the other hand, since the far-field array, i.e., the third array 130, has a thick width h3, an operation frequency f3 is low. In one exemplary embodiment, a plurality of piezoelectric elements each having widths different from each other may have the transmission and reception properties of the range where a plurality of frequencies exists.

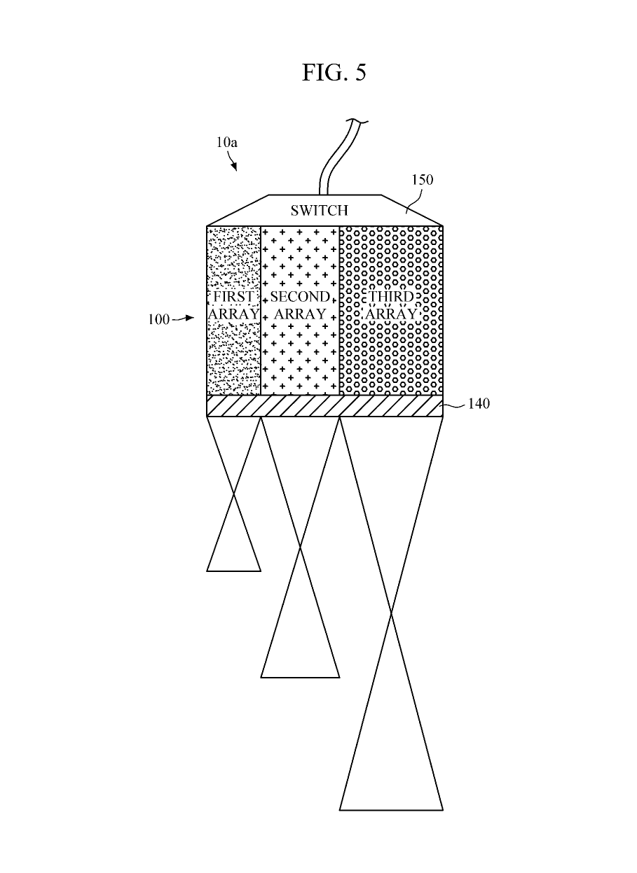

FIG. 5 is a diagram illustrating a constitution of an ultrasonic probe 10a having a switch 150 according to an exemplary embodiment.

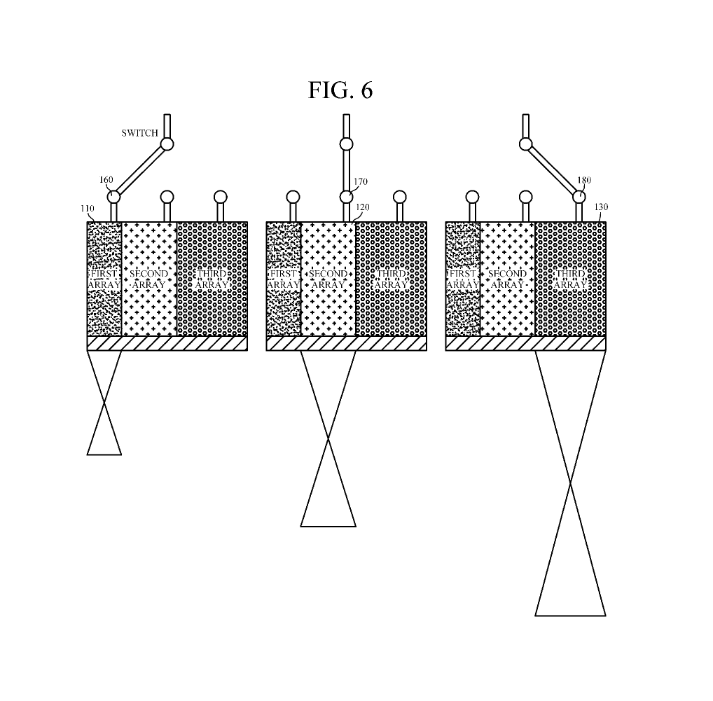

Referring to FIG. 5, the ultrasonic probe 10a includes arrays 100 and a switch 150. Since the arrays 100 each have widths different from each other in an elevation direction, their focal lengths are different from each other. The switch 150 selects one of the arrays and activates the selected array. The operation principle of the switch 150 will be specifically described in FIG. 6.

FIG. 6 is a diagram illustrating an operation principle of a switch 150 in FIG. 5 according to an exemplary embodiment.

Referring to FIGS. 5 and 6, the switch 150 selects one of the arrays and activates the selected array. For example, the switch 150 activates a first array 110 when a drive signal is applied to a first access point 160; a second array 120 when the drive signal is applied to a second access point 170; and a third array 130 when the drive signal is applied to a third access point 180.

As an example of using the switch 150 in a medical field, if a patient fat, so that ultrasonic waves need to penetrate deeply, a far-field array, i.e., the third array 130, is selected and activated by the switch 150. On the other hand, if the patient is slim, so that ultrasonic waves need to penetrate shallowly, a near-field array, i.e., the first array 110, is selected and activated through the switch 150. Here, the selection and activation of the corresponding array may include receiving an input of environment information of the object, analyzing the input information, and then selecting an array to be activated or being directly selected by an inspector. As described above, an optimum array is selected adaptively to an environment where the depth of a patient's target tissue changes, so that images with optimum qualities may be acquired.

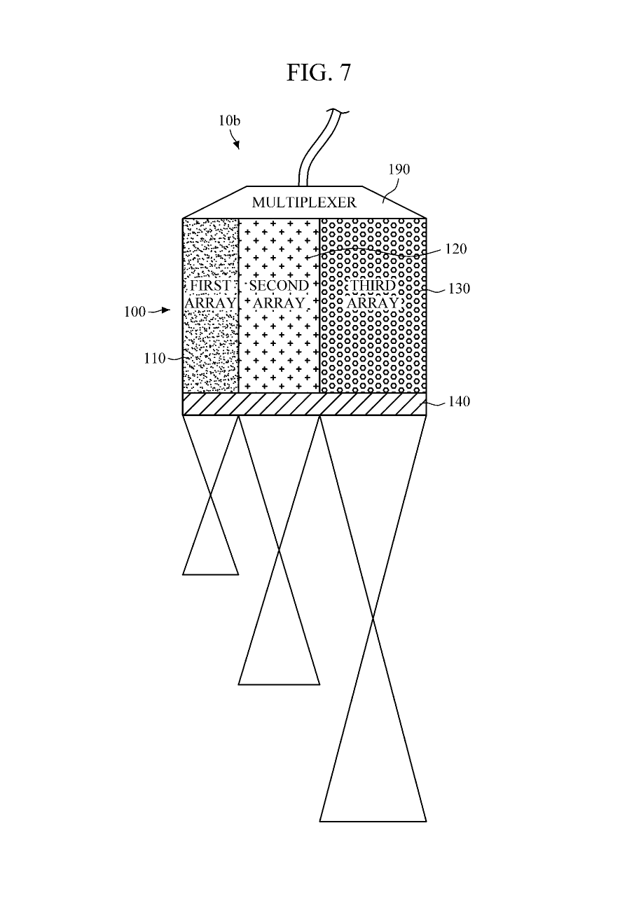

FIG. 7 is a diagram illustrating an ultrasonic probe 10b having a multiplexer according to an exemplary embodiment.

Referring to FIG. 7, an ultrasonic probe 10b includes arrays 100 and a multiplexer 190. Since the arrays 100 each have widths that are different from each other in an elevation direction, their focal lengths are different from each other. The multiplexer 190 activates all the arrays to control the activated arrays 100 to focus ultrasonic signals on each array. For example, as illustrated in FIG. 7, the multiplexer 190 simultaneously activates a first array 110, a second array 120, and a third array 130 to control the activated arrays 100 to focus ultrasonic signals on each of the arrays 110, 120, and 130.

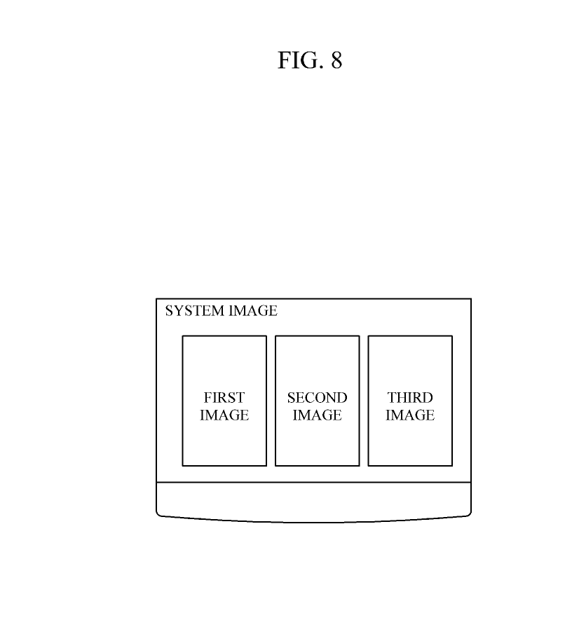

FIG. 8 is a diagram illustrating images acquired in a case where a multiplexer in FIG. 7 according to an exemplary embodiment is used.

Referring to FIGS. 7 and 8, an image is acquired for each array by using a multiplexer 190. For example, as illustrated in FIG. 8, in a case where three arrays are used, a first image, a second image and a third image may be acquired. In such a case, an inspector may select an optimum image among each of the images in consideration of a patient's circumstances.

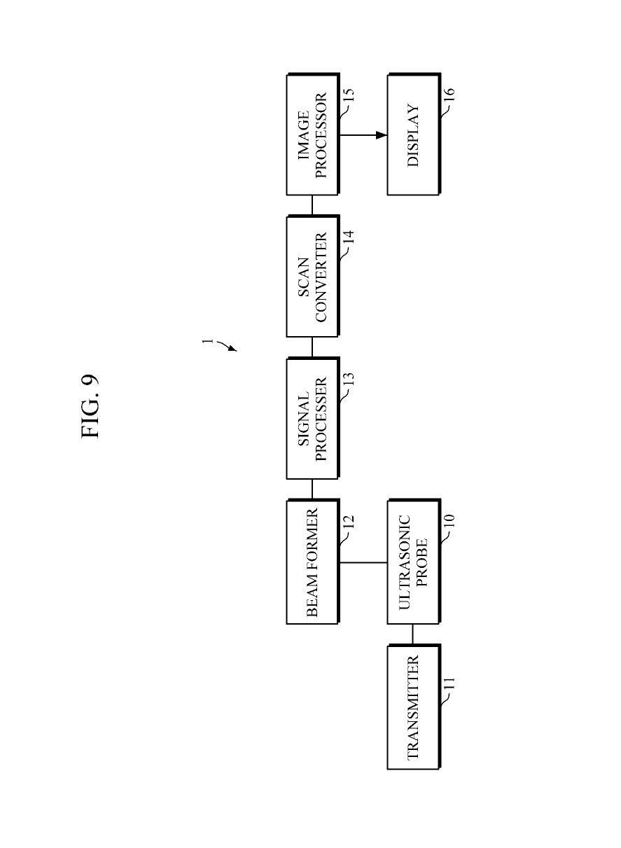

FIG. 9 is a diagram illustrating a constitution of an ultrasonic imaging diagnostic apparatus according to an exemplary embodiment.

Referring to FIG. 9, an ultrasonic imaging diagnostic apparatus 1 includes an ultrasonic probe 10, a transmitter 11, a beam former 12, a signal processor 13, a scan converter 14, an image processor 15, and a display 16. The ultrasonic imaging diagnostic apparatus 1 further includes storage, such as a memory (not illustrated).

The ultrasonic probe 10 includes at least one transducer element that performs an interconversion between an electrical signal and an ultrasonic signal. The transducer element generates an ultrasonic signal in response to the electrical signal, and includes a piezoelectric element to generate an electrical signal in response to an ultrasonic echo signal. A transmission ultrasonic beam, which is output from each transducer element in response to the electrical signal, may be shown as having high-frequency or low-frequency properties according to properties of the piezoelectric element.

An ultrasonic probe 10 according to an exemplary embodiment includes: a plurality of arrays, of which focal lengths are different from each other due to their widths that are different from each other in an elevation direction; and includes a switch that selects one of the plurality of arrays and activates the selected array.

An ultrasonic probe 10 according to another exemplary embodiment includes: a plurality of arrays, of which focal lengths are different from each other due to their widths that are different from each other in an elevation direction; and includes a multiplexer that simultaneously activates the plurality of arrays and controls the activated arrays to focus ultrasonic signals on each array.

The ultrasonic probe 10 transmits an ultrasonic signal to an object through the transmitter 11 by responding to an electrical transmission signal, which is output from a transmission signal generator (not illustrated), and receives an echo signal that has been reflected from the object. The ultrasonic probe 10 outputs an electrical reception signal in response to the received echo signal.

The beam former 12 forms a reception focusing beam by receiving and focusing the reception signal that has been output from the ultrasonic probe 10; and the signal processor 13 forms ultrasonic imaging data by performing an envelope detection treatment, etc., with respect to the reception focusing beam that has been output from the beam former 12.

The scan converter 14 converts the ultrasonic imaging data, output from the signal processor 13, to a data format that enables the ultrasonic imaging data to be displayed; and the image processor 15 processes the imaging data, output from the scan converter 14, and then transmits the processed data to the display 16, which then displays the image received from the image processor 15.

While this invention has been particularly shown and described with reference to preferred embodiments thereof, it will be understood by those skilled in the art that various changes in form and details may be made therein without departing from the spirit and scope of the invention as defined by the appended claims. The preferred embodiments should be considered in descriptive sense only and not for purposes of limitation. Therefore, the scope of the invention is defined not by the detailed description of the invention but by the appended claims, and all differences within the scope will be construed as being included in the present invention.

1. An ultrasonic probe, comprising:
a first array;
a second array configured to be connected to the first array in parallel in an elevation direction, and have a focal length that is different from a focal length of the first array due to a width that is different from a width of the first array in an elevation direction; and
a switch configured to select one of the first and second arrays and activate the selected array.

2. The ultrasonic probe of claim 1, wherein the first array is a near-field array with a width h1 and a focal length L1, and the second array is a far-field array with a width h2 and a focal length L2, where h1 is smaller than h2 and L1 is smaller than L2.

3. The ultrasonic probe of claim 2, wherein the switch is configured to operate the first array at a high frequency, and the second array at a low frequency.

4. The ultrasonic probe of claim 1, wherein the switch is configured to in response to an array drive signal being applied to a first access point, activate the first array, and in response to the array drive signal being applied to a second access point, activate the second array.

5. The ultrasonic probe of claim 1, wherein each array is configured to have a concave surface that is positioned in an axial direction in which beams proceed.

6. The ultrasonic probe of claim 5, further comprising:
an acoustic lens configured to focus, on an inside of an object, an ultrasonic signal generated from the array activated by the switch, wherein the acoustic lens is configured to be a plane.

7. An ultrasonic probe, comprising:
a first array;
a second array configured to be connected to the first array in parallel in an elevation direction, and have a focal length that is different from a focal length of the first array due to a width that is different from a width of the first array in an elevation direction; and
a multiplexer configured to simultaneously activate the first and second arrays and control the activated arrays to focus ultrasonic signals on each array.

8. The ultrasonic probe of claim 7, wherein the first array is a near-field array with a width h1 and a focal length L1, and the second array is a far-field array with a width h2 and a focal length L2, where h1 is smaller than h2 and L1 is smaller than L2.

9. The ultrasonic probe of claim 8, wherein the multiplexer is configured to operate the first array at a high frequency, and the second array at a low frequency.

10. The ultrasonic probe of claim 7, wherein each array is configured to have a concave surface that is positioned in an axial direction in which beams proceed.

11. The ultrasonic probe of claim 10, further comprising:
an acoustic lens configured to focus, on an inside of an object, the ultrasonic signal generated from the array activated by the multiplexer, wherein the acoustic lens is configured to be a plane.

12. An ultrasonic imaging diagnostic apparatus, comprising:
an ultrasonic probe, which comprises a plurality of arrays each having different focal lengths due to different widths in an elevation direction, and which comprises a switch configured to select one of the plurality of arrays and activate the selected array;
a transceiver configured to transceive an ultrasonic signal to or from an object through the array selected and activated by the ultrasonic probe;
an image processor configured to in response to a reflected ultrasonic signal being received from the object by the transceiver, generate a displayable image by using the received reflected ultrasonic signal; and
a display configured to display the image generated by the image processor.

13. The ultrasonic imaging diagnostic apparatus of claim 12, wherein the ultrasonic probe comprises a near-field array with a width h1 and a focal length L1, and a far-field array with a width h2 and a focal length L2, where h1 is smaller than h2, and L1 is smaller than L2.

14. An ultrasonic imaging diagnostic apparatus, comprising:
an ultrasonic probe, which comprises a plurality of arrays each having different focal lengths due to different widths in an elevation direction, and which comprises a multiplexer configured to simultaneously activate the plurality of arrays and control the activated arrays to focus ultrasonic signals on each array;
a transceiver configured to transceive an ultrasonic signal to or from an object through the array activated by the ultrasonic probe;
an image processor configured to in response to a reflected ultrasonic signal being received from the object by the transceiver, generate a displayable image by using the received reflected ultrasonic signal; and
a display configured to display the image generated by the image processor.

15. The ultrasonic imaging diagnostic apparatus of claim 14, wherein the ultrasonic probe comprises a near-field array with a width h1 and a focal length L1, and a far-field array with a width h2 and a focal length L2, where h1 is smaller than h2, and L1 is smaller than L2.
